(19) United States
(12) Patent Application Publication
(10) Pub. No.: US 2026/0256478 A1
(43) Pub. Date: Sep. 3, 2026

(54) SYSTEMS, APPARATUSES, AND METHODS ORTHOPEDIC SURGERY

(71) Applicant: Forma Medical, Inc., Camp Hill, PA (US)

(72) Inventor: Christopher F. Hyer, Worthington, OH (US)

(73) Assignee: Forma Medical, Inc., Camp Hill, PA (US)

(21) Appl. No.: 18/879,691

(22) PCT Filed: Jun. 30, 2023
(86) PCT No.: PCT/US23/26728

§ 371 Date: Dec. 27, 2024

Related U.S. Application Data
(60) Provisional application No. 63/357,435, filed on Jun. 30, 2022.
(60) Provisional application No. 63/357,443, filed on Jun. 30, 2022.
(60) Provisional application No. 63/357,456, filed on Jun. 30, 2022.
(60) Provisional application No. 63/357,453, filed on Jun. 30, 2022.
(60) Provisional application No. 63/357,462, filed on Jun. 30, 2022.
(60) Provisional application No. 63/357,467, filed on Jun. 30, 2022.
(60) Provisional application No. 63/357,480, filed on Jun. 30, 2022.

(51) Int. Cl. A61B 17/17; A61B 17/00; A61B 17/56; A61B 17/90

(52) U.S. Cl. CPC A61B 17/1775; A61B 17/90; A61B 17/00234; A61B 2017/00238; A61B 2017/565

(57) ABSTRACT

Multi-function targeting guides for minimally invasive 1st metatarso-phalangeal (MTP) joint fusion or arthrodesis configured to be positioned with a superficial anatomy-based surface match to establish precise positioning for surgical cuts, drilling, burring, and orthopedic screw affixation. Surgical apparatuses, namely bone reamers, reamer guides, burr sleeves, and positioners to assist in the joint fusion procedure. Methods of using the multi-functional targeting guides and surgical apparatus in conducting a MTP arthrodesis procedure.

### CROSS-REFERENCE TO RELATED APPLICATIONS

This application is a national phase application filed under 35 U.S.C. § 371 of PCT Application No. PCT/US2023/26728, filed on Jun. 30, 2023, which claims priority to, and the benefit under 35 U.S.C. § 119(e) of, U.S. Provisional Application Nos. 63/357,435, filed on Jun. 30, 2022; 63/357,443, filed on Jun. 30, 2022; 63/357,456, filed on Jun. 30, 2022; 63/357,453, filed on Jun. 30, 2022; 63/357,462, filed on Jun. 30, 2022; 63/357,467, filed on Jun. 30, 2022; 63/357,480, filed on Jun. 30, 2022; the entire contents of all of which are incorporated herein by reference.

### FIELD OF THE INVENTION

The present disclosure relates generally to systems and methods for minimally invasive 1^{st} metatarso-phalangeal (MTP) joint fusion or arthrodesis utilizing a multi-function targeting guides.

### BACKGROUND OF THE INVENTION

Orthopedic procedures, such as minimally invasive surgery or minimal incision surgery (MIS) fusion or arthrodesis of the 1^{st} metatarso-phalangeal (MTP), is a surgical procedure used to treat and correct painful disorders or diseases of the 1^{st} MTP joint. For example, it is used to treat arthritis in the 1^{st} MTP joint by removing the degraded part of the joint and fusing the 1^{st} metatarsal and phalangeal bones together at the distal and proximal ends thereof, respectively. A result of the MTP joint fusion is to establish of stable, plantigrade first toe and has a high degree of both functional and physiological success.

There are many different type of MTP joint fusion plates that are known in the art and commercially available. For example, U.S. Pat. No. 8,167,918 discloses an MTP joint fusion plate. Similarly, U.S. Pat. No. 9,301,790 discloses a cannulated orthopedic fixation screw. An example of a known MTP joint fusion plating platform and screw system are the ANCHORAGE CP plating system and the ASNIS MICRO screw system (Stryker Corporation, Kalamazoo, Michigan).

There is therefore a need to provide multi-function targeting guides that are positioned with a superficial anatomy-based surface match to establish precise positioning for surgical cuts, drilling, burring, and orthopedic screw affixation. There is a further need to provide surgical apparatuses, namely bone reamers, reamer guides, burr sleeves, and positioners to assist in the MIS-MTP arthrodesis procedure. There is yet a further need to provide methods of using the multi-functional targeting guides and surgical apparatus in conducting an MIS-MTP arthrodesis procedure.

For exemplary purposes only, the present disclosure will refer to MIS-MTP procedures. Such example is intended and should be construed as non-limiting of the scope of the disclosure. Rather, the disclosure is intended to be broadly construed and limited only by the scope of the claims appended hereto.

### SUMMARY OF THE INVENTION

An embodiment of the present disclosure includes a surgical reamer. The surgical reamer includes a flexible wire having a proximal end and a distal end, the proximal end configured to removably couple to a surgical drill. The surgical reamer further includes a coupling at the distal end thereof. The surgical reamer further includes a reamer tip at the distal end of the flexible wire.

A further embodiment of the present disclosure includes a surgical reamer. The surgical reamer includes a flexible wire having a proximal end and a distal end, the proximal end configured to removably couple to a surgical drill. The surgical reamer further includes a coupling at the distal end thereof. The surgical reamer further includes a reamer tip at the distal end of the flexible wire.

A further embodiment of the present disclosure includes a surgical reamer guide. The surgical reamer guide includes a tubular sheath having an angular displacement between about 30 to about 45 degrees along its longitudinal axis. The tubular sheath includes a central lumen extending its entire longitudinal axis. The central lumen is configured to receive a flexible wire reamer to pass there through and permit free rotation of the flexible wire reamer therein.

A further embodiment of the present disclosure includes a metatarsal-phalangeal targeting guide. The metatarsal-phalangeal targeting guide includes a chip having proximal and distal aspects thereof. The chip has medial or lateral surfaces thereof configured to approximate a medial or lateral skin surface of a metatarsal bone of a 1^{st} metatarsal-phalangeal joint. The metatarsal-phalangeal targeting guide includes at least two openings passing through the medial or lateral surfaces and a thickness of the chip, each of the at least two openings having different angular opening axes and planes.

A further embodiment of the present disclosure includes a method for minimally invasive incision surgery to fuse the first metatarsal-phalangeal joint. The method includes exposing the first metatarsal-phalangeal joint. The method further includes pinning a guide chip to a medial or lateral surface of a first metatarsal bone. The method further includes drilling non-intersecting screw channels in each of the phalangeal and first metatarsal bones across the first metatarsal-phalangeal joint using the guide chip to drill the non-intersecting screw channels. The method further includes affixing the first metatarsal-phalangeal joint in a desired position with compression of the joint.

A further embodiment of the present disclosure includes a modular positioner. The modular positioner includes a mid-foot heel cup component having an open mesh configuration configured to permit fixation screws and wires to pass through the open mesh and shaped to cup and secure a patients heel and mid-foot therein.

A further embodiment of the present disclosure includes a method for positioning a great toe for minimally invasive incision surgery to fuse the first metatarsal-phalangeal joint. The method includes removably placing a patient's heel in a heel cup, the heel cup having an open mesh construction and couplings to removably couple a great toe positioner. The method further includes placing a toe positioner having an open mesh construction such that it abuts a plantar surface of the great toe. The method further includes removably coupling the toe positioner to the heel cup and positioning the patient's great toe in a desired position for the joint fusion procedure.

A further embodiment of the present disclosure includes a metatarsal-phalangeal targeting guide. The metatarsal-phalangeal targeting guide includes a chip having proximal and distal aspects thereof, the chip having a medial or lateral surfaces thereof configured to approximate a medial or lateral skin surface of a proximal phalanx bone of a 1^{st} metatarsal-phalangeal joint. The metatarsal-phalangeal targeting guide further includes at least two openings passing through the medial or lateral surfaces and a thickness of the chip, each of the at least two openings having different angular opening axes and planes.

A further embodiment of the present disclosure includes a method for minimally invasive incision surgery to fuse the first metatarsal-phalangeal joint. The method includes exposing the first metatarsal-phalangeal joint. The method further includes pinning a guide chip to a medial or lateral surface of a proximal phalanx bone. The method further includes drilling non-intersecting screw channels in each of the phalangeal and first metatarsal bones across the first metatarsal-phalangeal joint using the guide chip to drill the non-intersecting screw channels. The method further includes affixing the first metatarsal-phalangeal joint in a desired position with compression of the joint.

A further embodiment of the present disclosure includes a bone drilling guide. The bone drilling guide includes a chip having proximal and distal aspects thereof, the chip having a medial or lateral surfaces thereof configured to approximate a medial or lateral skin surface over a joint. The bone drilling guide further includes at least two openings passing through the medial or lateral surfaces and the thickness of the chip, each of the at least two openings having different angular opening axes in different planes.

A further embodiment of the present disclosure includes a metatarsal-phalangeal targeting guide. The metatarsal-phalangeal targeting guide includes at least one chip having proximal and distal aspects thereof. Each of the at least one chip has at least one opening passing through medial or lateral surfaces and a thickness of the chip. Each of the at least one opening is configured to guide drilling of a screw channel into a first bone and a second bone across a metatarsal-phalangeal joint in different non-intersecting planes.

A further embodiment of the present disclosure includes a metatarsal-phalangeal targeting guide. The metatarsal-phalangeal targeting guide includes a first chip having proximal and distal aspects thereof, and at least one opening passing through medial or lateral surfaces and a thickness of the chip. The at least one opening of the first chip has a first opening angle through the first chip. The metatarsal-phalangeal targeting guide further includes a second chip having proximal and distal aspects thereof, and at least one opening passing through medial or lateral surfaces and a thickness of the chip. The at least one opening of the second chip has a second opening angle through the first chip such that the first opening angle and the second opening angle have non-intersecting planes within each of the first chip and the second chip.

A further embodiment of the present disclosure includes a method for minimally invasive incision surgery to fuse the first metatarsal-phalangeal joint. The method includes exposing the first metatarsal-phalangeal joint. The method further includes pinning a guide chip across at least a portion of the first metatarsal-phalangeal joint. The method further includes drilling non-intersecting screw channels in overlapping planes in each of the phalangeal and first metatarsal bones. The method further includes affixing screws in each of the non-intersecting screw channels across the first metatarsal-phalangeal joint thereby compressing of the joint for fusion.

A further embodiment of the present disclosure includes an interphalangeal joint targeting guide. The interphalangeal joint targeting guide includes a main body member having a longitudinal axis and a targeting pin projecting distally from the main body member along a mid-line of the main body and co-axial with the longitudinal axis of the main body.

### BRIEF DESCRIPTION OF THE DRAWINGS

FIG. 1 is a top plan view of a first variant of a targeting guide in accordance with the present disclosure;

FIG. 2 is a cross-sectional view taken along line 2-2 of FIG. 1;

FIG. 3 is a top plan view of a second variant of a targeting guide in accordance with the present disclosure;

FIG. 4 is a top plan view of a third variant of a targeting guide in accordance with the present disclosure;

FIG. 5 is a perspective view of the third variant of the targeting guide in accordance with the present disclosure;

FIG. 6 is a cross-sectional view taken along line 6-6 of FIG. 5;

FIG. 7 is a side elevational view of an alignment carriage in accordance with the present disclosure;

FIG. 8 is a top plan view of a fourth variant of a targeting guide in accordance with the present disclosure;

FIG. 9 is a top plan view of a fifth variant of a targeting guide in accordance with the present disclosure;

FIG. 10 is a top plan view illustrating misalignment of a proximal phalanx and a distal phalanx typical of hallux interphalangeus;

FIG. 11A is a sixth variant of a targeting guide in accordance with the present disclosure;

FIG. 11B is a top plan view of the sixth variant of the targeting guide in association with the misaligned proximal phalanx and distal phalanx for treating hallux interphalangeus;

FIG. 11C is a cross-sectional view taken along line 11C-11C of FIG. 11B;

FIG. 12A is a top plan view of a two-piece variant of a targeting guide in accordance with the present disclosure;

FIG. 12B is a top plan view of the two-piece variant of the targeting guide in a partially engaged state;

FIG. 12C is an exploded view of the two-piece variant of the targeting guide;

FIG. 13 is a fragmentary side elevational view of a flexible reamer bit in accordance with the present disclosure;

FIG. 14 is a side elevational view of a reamer tube instrument in accordance with the present disclosure;

FIG. 15 is a perspective view of a modular positioner in accordance with the present disclosure;

FIG. 16 is a perspective view of a heel component of the modular positioner in accordance with the present disclosure;

FIG. 17 is a perspective view of a 1^{st} toe component of the modular positioner in accordance with the present disclosure;

FIG. 18 is a perspective view of a forefoot component of the modular positioner in accordance with the present disclosure;

FIG. 19 is a perspective view of the burr sleeve engaged upon a reamer bit in accordance with the present disclosure;

FIG. 20 is a cross-sectional view taken along line 20-20 of FIG. 19;

FIG. 21 is a perspective view of the burr sleeve engaged upon a reamer bit placed into an opening of a targeting guide illustrating sweeping motion of the reamer bit and burr sleeve in the targeting guide;

FIG. 22 is a cross-sectional view taken along line 22-22 of FIG. 19;

FIG. 23 is a transverse cross-sectional view illustrating an alternative version of a central lumen of the burr sleeve; and

FIG. 24 is a transverse cross-sectional view illustrating a multi-lumen variant of the burr sleeve.

### DETAILED DESCRIPTION OF THE PREFERRED EMBODIMENTS

For purposes of clarity, the following terms used in this patent application will have the following meanings:

The terminology used herein is for the purpose of describing example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the order discussed or illustrated, unless specifically identified as an order of performance. It is also to be understood that additional or alternative steps may be employed.

When an element or layer is referred to as being “on,” “engaged,” “connected,” or “coupled” to or with another element, it may be directly on, engaged, connected or coupled to the other element or layer, or intervening elements or layers may be present. In contrast, when an element is referred to as being “directly on,” “directly engaged to,” “directly connected to,” or “directly coupled to” or with another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below”, or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

“Substantially” is intended to mean a quantity, property, or value that is present to a great or significant extent and less than, more than or equal to total. For example, “substantially vertical” may be less than, greater than, or equal to completely vertical.

“About” is intended to mean a quantity, property, or value that is present at ±10%. Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. In addition, disclosure of ranges includes disclosure of all values and further divided ranges within the entire range, including endpoints given for the ranges.

Recitation of ranges of values herein are merely intended to serve as a shorthand method of referring individually to each separate value falling within the recited range, unless otherwise indicated herein, and each separate value is incorporated into the specification as if it were individually recited herein.

References to “embodiment” or “variant”, e.g., “one embodiment,” “an embodiment,” “example embodiment,” “various embodiments,” etc., may indicate that the embodiment(s) or variant(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment or variant, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

The term “material” is intended to refer to encompass biocompatible materials, including metals, ceramics, plastics, composites, and combinations or hybrids thereof.

As used in this application the term “layer” is intended to mean a substantially uniform material limited by interfaces between it and adjacent other layers, substrate, or environment.

The terms “circumferential” or “circumferential axis” is intended to refer to the radial direction of a tubular, cylindrical or annular material or to the Y-axis of a polygonal material.

The terms “longitudinal,” “longitudinal axis,” or “tube axis” are intended to refer to an elongate aspect or axis of a material or to the X-axis of the material.

The term “medial” is intended to denote a position towards the midline of the body.

The term “lateral” is intended to mean a position away from the midline of the body.

The term “plantar” is intended to refer to a position toward the sole of the foot.

The term “dorsal” is intended to refer to a position away from the sole of the foot.

The present disclosure pertains generally to systems, apparatuses, and methods for orthopedic surgery, particularly for lower and upper extremities such as foot and ankle surgery. For example, the present disclosure pertains to systems, apparatuses, and methods useful in, for example, minimally invasive surgery or minimal incision surgery (MIS) fusion or arthrodesis of the 1^{st} metatarso-phalangeal (MTP) joint. More specifically, the present disclosure pertains to multi-function targeting guides which may be configured to be positioned with a superficial anatomy-based surface match to establish precise positioning for surgical cuts, drilling, burring, and orthopedic screw affixation. Further, the present disclosure pertains to surgical apparatuses, namely bone reamers, reamer guides, burr sleeves, and positioners to assist in the orthopedic surgical procedures. Finally, the present disclosure pertains to methods of using the multi-functional targeting guides and surgical apparatus in conducting an orthopedic surgical procedures, for example, MIS-MTP arthrodesis procedure.

Turning now to the accompanying Figures, there is illustrated the variants of the present disclosure pertaining to the multi-function targeting guides that are positioned with a superficial anatomy-based surface match to establish precise positioning for surgical cuts, drilling, burring, and orthopedic screw affixation; the variants of the present disclosure pertaining to the surgical apparatuses, namely bone reamers, reamer guides, burr sleeves, and positioners to assist in the MIS-MTP arthrodesis procedure; and the methods of using the multi-functional targeting guides and surgical apparatus in conducting an MID-MTP arthrodesis procedure.

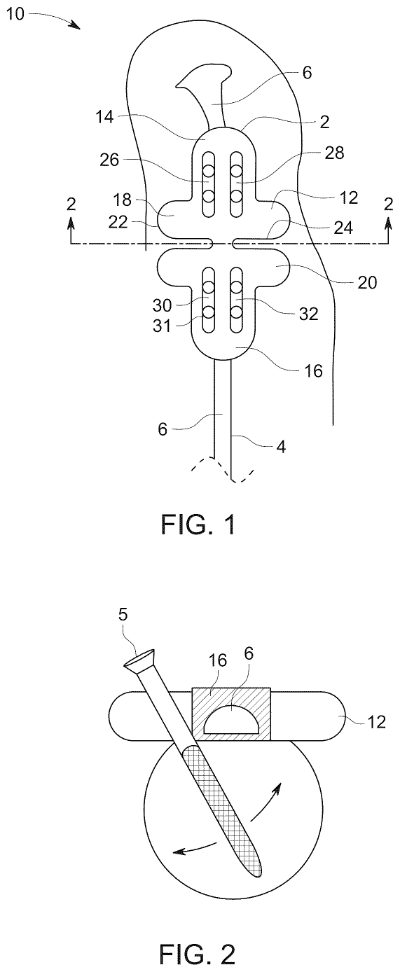

With particular reference to FIGS. 1 and 2, there is illustrate a multi-function targeting guide 10 having a main body 12 having a plurality of elongate openings 22, 24, 26, 28, 30, 32 passing through the main body from a dorsal surface to a plantar surface thereof. The main body 12 is illustrated in the Figures as having a generally cruciform shape with first and second longitudinal extensions 14, 16 and first and second lateral extensions 18, 20 projections extending laterally from the targeting guide 10. Those skilled in the art will appreciate that the illustrated generally cruciform shape is intended to be exemplary and not intended to be limiting as the only shape of the main body. Rather, the main body 12 may be generally polygonal, elliptical, ovular, or other geometric shape that has the plurality of elongate openings passing through that are positioned as hereinafter described. Optionally, the main body 12 may have a shallow concave curvature on the plantar surface of the main body to surface-match the skin-surfaces of the MTP joint.

A plurality of first openings 26, 28, 30, 32 pass through the main body 12 of the targeting guide and positioned laterally and medially from a central longitudinal axis of the main body 12. Each of the plurality of first openings 26, 28, 30, 32 pass entirely through the main body 12. The plurality of first openings 26, 28, 30, 32 may be each be an elongate slot or a plurality of openings arrayed along a longitudinal axis of the body member and laterally and medially spaced from the mid-line of the body member. A plurality of second openings 22, 24 are provided that pass through the main body 12 of the targeting guide and are oriented substantially perpendicular to the mid-line of the body member. Each of the plurality of second openings 22, 24 are preferably elongated slots and pass through the main body 12 and are open at both the dorsal and plantar surfaces of the body member. Each of the elongate slots 22, 24 have both a slot width and a slot length that are configured to allow fixation wires, drills, burrs, or other instruments to pass into and through the elongate slots. In particular, the elongate slots 22, 24 have a slot width and slot length that permit a burr to sweep across the articular aspects of the metatarsal and phalangeal bones to make the necessary cuts to allow formation of mating surfaces on the respective bones.

The plantar surface of the main body 12 has a shallow concave curvature and a channel formed into the plantar surface. The channel in the plantar surface of the main body 12 serves two functions: i) to accommodate and protect the Extensor hallucis longus (EHL) tendon 6, that passes substantially midline over the MTP joint, and ii) to center the main body 12 over the approximate mid-line of the joint and over the EHL tendon 6. As the EHL has a substantially round transverse cross-section at the MTP joint, it is preferable that the channel have a semi-circular or triangular configuration such that the EHL tendon 6 is nested within the channel and protected from being interfered with or damaged during manipulation of instruments through the plurality of second openings 22, 24 during the arthrodesis procedure.

The plurality of first openings 26, 28, 30, 32 are configured to allow a fixation wire, such as a Kirschner wire (K-wire) or olive wire, to be placed through the elongate openings to secure the main body to both the distal aspect of the 1^{st} metatarsal bone and the proximal aspect of the phalangeal bone, while allowing longitudinal adjustment of the main body 12 along a longitudinal axis of the joint and to position the elongate slots in the first and second longitudinal extension over the articular surfaces of the MTP joint. Each of the plurality of second openings 22, 24 are configured to allow different size burrs to pass into and through the elongate openings and allow surgical access to the articular surfaces and ends of each of the first metatarsal bone and the phalangeal bone, as shown in FIG. 2.

MTP joint arthrodesis is typically performed using either what is conventionally known as a flat cut or a “cup and cone” cut. In the flat cut the distal aspect of the metatarsal bone and the proximal aspect of the phalangeal bone are both planarized to have mating surfaces. In the “cup and cone” cut, the distal aspect of the metatarsal bone is cut with a convex radius and the proximal aspect of the phalangeal bone is cute with a concave radius that mates with the convex surface of the metatarsal bone to allow bone growth between the metatarsal bone and the phalangeal bone.

For flat cut MTP joint fusion, i.e., where each of the joint surfaces of the first metatarsal bone and the phalangeal bone are to be cut with planar mating surfaces, the elongate openings 22, 24, 26, 28, 30, 32 will have a substantially perpendicular orientation relative to the longitudinal axis of the main body 12 of the targeting guide 10.

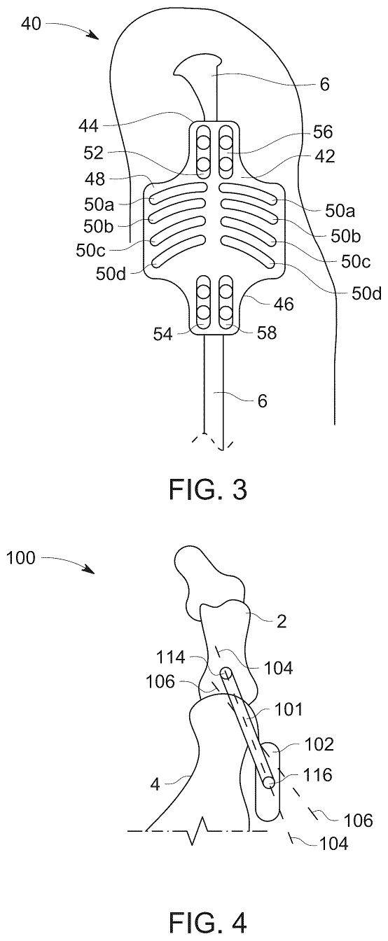

As depicted in FIG. 3, for the “cup and cone” arthrodesis, the targeting guide 40 is configured to facilitate both concave and convex shaping of the MTP joint surfaces. The targeting guide 40 includes a main body (not numbered) having a distal portion 44, a main body portion 48, and proximal portion 46 opposite the distal portion 44. The proximal and distal portions 44, 46 have a lateral width that is less than the lateral width of the main body portion 48. The distal portion includes include a plurality of bores or holes 52, 56 spaced apart along either side of a longitudinal axis (not numbered). The bores 52 and 56 are arranged in series along a longitudinal direction that is parallel to the longitudinal axis. The bores 52 and 56 are arranged in series along longitudinal direction. The proximal portion includes similar bores or holes 54, 58 spaced apart along either side of a longitudinal axis (not numbered). The bores 54 and 58 are arranged in series along a longitudinal direction that is parallel to the longitudinal axis. The bores 54 and 58 are arranged in series along longitudinal direction. The bores 52 and 54 may be aligned with each other and the bores 44 and 54 are aligned with each. The main body portion 48 includes a first plurality of elongate slots 50a-50d on one side of the central axis and second plurality of elongate slots 50a-50 on the other side of the longitudinal axis. The slots 50a-50d include graduated opening widths and/or graduated angular positions relative to the longitudinal axis of the targeting guide main body. The graduated opening widths and/or graduated angular positions are configured to permit cutting different curvatures in each of the metatarsal and phalangeal bones. The slots 50a-50d each having slight curvature and curve in generally proximal direction toward the proximal portion 56. In the example shown, there are four elongated slots on one side of the longitudinal axis and four elongated slots on the other side of the longitudinal axis.

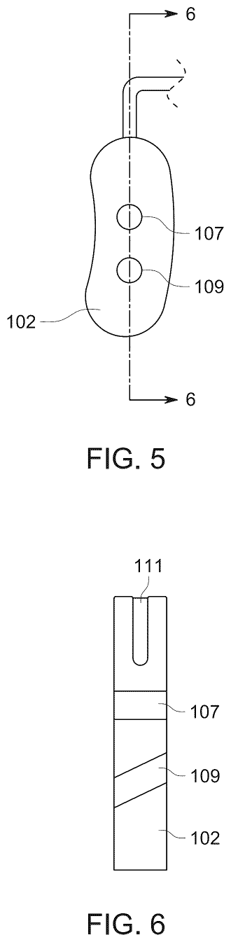

Other variants of a targeting guide 100 are shown in FIGS. 4 to 9. The targeting guide variants of FIGS. 4 to 9 are configured to be removably attached to a lateral or medial aspect of the superficial anatomy of the MTP joint. Each of the targeting guide variants has a surface configured to match the anatomic superficial surface profile of the MTP joint. A plurality of bores pass through each of the targeting guide variants with different angular orientations relative to the superficial matching surface of each targeting guide. A first bore is configured as a drill guide and fixation screw guide having a first angular orientation. A second bore is also configured as a drill guide and fixation guide and has a second angular orientation different from the first angular orientation of the second bore. It is important that the first and second angular orientations of the first and second bores, respectively, be oriented such that when fixation screws are inserted into the drilled bores, the fixation screws do not intersect or interfere with each other and the joint is brought into close approximation to allow for bone growth and fusion between the metatarsal and phalangeal bones 4, 2. A third bore is configured to accept a fixation wire, such as a K wire or olive wire that both retains the position of the targeting guide and the position of the MTP joint while the fusion procedure is being performed.

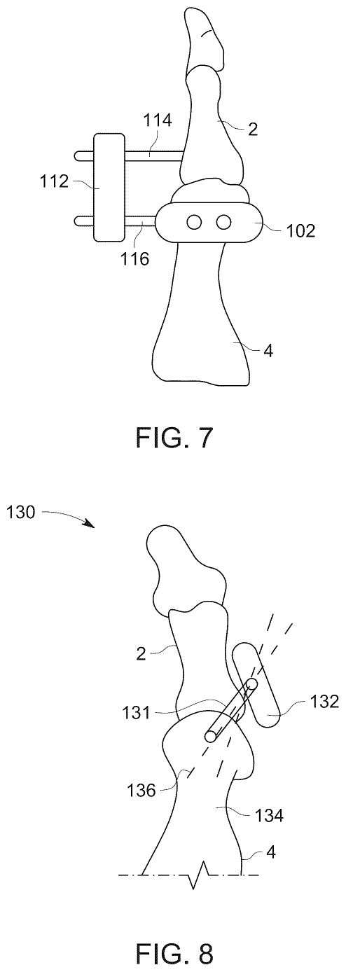

As shown in FIG. 7, an alignment carriage may be employed for placement of the fixation screws in the bores drilled into the metatarsal bone 4 or phalangeal bone 2. The alignment carriage couples to a pin placed mid-line in the phalangeal bone 2 or metatarsal bone 4 that serves as a reference point for aligning the fixation screws with the bores drilled into the metatarsal and/or phalangeal bones 4, 2. The alignment carriage includes a carriage floor 114, a carriage arm 116 parallel to the carriage floor 114, the carriage arm 116 has a carriage arm plate 102 having two openings 107, 109 passing laterally through the carriage arm plate 102, and a connecting arm 112 spanning the carriage floor 114 and the carriage arm 116 and maintaining the carriage floor 114 and carriage arm 116 in a space apart and adjustable relationship. One opening 109 in the carriage arm plate is angled about 45 degrees relative to the longitudinal axis of the MTP joint and a second opening 107 in the carriage arm is angled about 30 degrees relative to the longitudinal axis of the MTP joint. In this manner, fixation screws placed through the two openings 107, 109 in the carriage arm 116 will pass into and through the MTP joint at different and non-interfering angles. It will be understood by those skilled in the art that the guide carriage may be oriented to have either a phalangeal approach or a metatarsal approach.

The targeting guide 100 may be positioned at the metatarsal bone 4 as depicted in FIG. 4 with the wire 104 passing through the phalangeal bone 2 being joined to the carriage arm 116 at one end thereof that serves as a reference point for aligning the carriage arm plate 102 with the out of plane bores drilled in either the phalanx or metatarsal bones. Once the carriage arm plate 102 is aligned with the drilled bores, the fixation screws are passed through the openings 107, 109 in the carriage arm plate and into the metatarsal bone 4 and into the phalangeal bones 2. Once the fixation screws are placed, the carriage arm 116, the wires or pins 104, and the carriage arm plate 102 are removed.

Alternatively, as illustrated in FIG. 8, the targeting guide 130 may be positioned at the phalangeal bone 2, with the wire 134 passing through the metatarsal bone 4 and the fixation screws passing through the phalangeal bone 2 and into the metatarsal bone 4. The targeting guides illustrated in FIGS. 4 and 8 may be a single targeting guide configured to be reversible to accommodate a metatarsal approach and a phalangeal approach. Alternatively, the targeting guides may be configured to have dedicated application for only the metatarsal bone 4 or to the phalangeal bone 2.

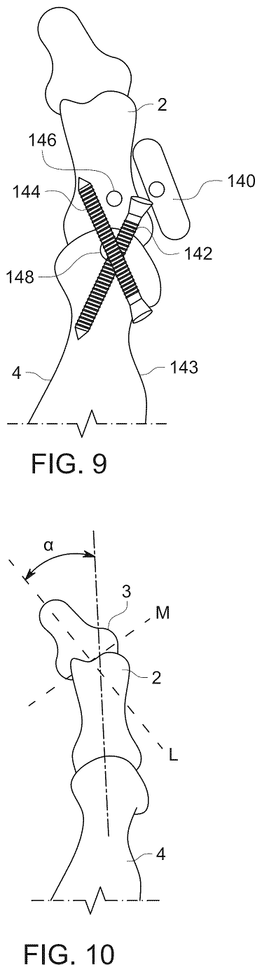

FIG. 9 is yet another alternative variant of the targeting guide 130 of the present disclosure. In this variant, the targeting guide 130 extends across the MTP joint and has two bores 142, 144 at each of the proximal and distal aspects of the targeting guide that are oriented approximately 45 degrees relative to the longitudinal axis of the toe, such that the drill bores and the fixation screws pass into the bones at approximately a 45 degree angle relative to the longitudinal axis of the toe. The two bores are out of plane relative to each other so that the drill bores and the fixation screws do not intersect.

With this variant of the targeting guide 130, two guidewire or olive wire holes 146, 148 are placed in each of the proximal phalanx and distal metatarsal at substantially midline positions on the joint, and a guide wire or olive wire is placed to secure the targeting guide position relative to the MTP joint. A phalangeal bore 144 passes through the targeting guide at approximately a 45 degree angle relative to the longitudinal axis of the phalanx. A metatarsal bore 142 passes through the targeting guide at approximately a 45 degree angle relative to the longitudinal axis of the metatarsal bone. The phalangeal bore 144 and the metatarsal bore 142 are out of plane relative to each other so that the bores do not intersect and the fixation screws, when placed within each of the phalangeal bore and the metatarsal bore, do not intersect or interfere with each other. Once the screws are placed, the guidewire or olive wires are removed and replaced with a staple or other fixation device placed in the guidewire or olive wire holes that spans the joint.

The targeting guide 130 may be configured to also position and drill the guidewire or olive wire holes. Alternatively, the guidewire or olive wire holes may be made using a separate reference guide.

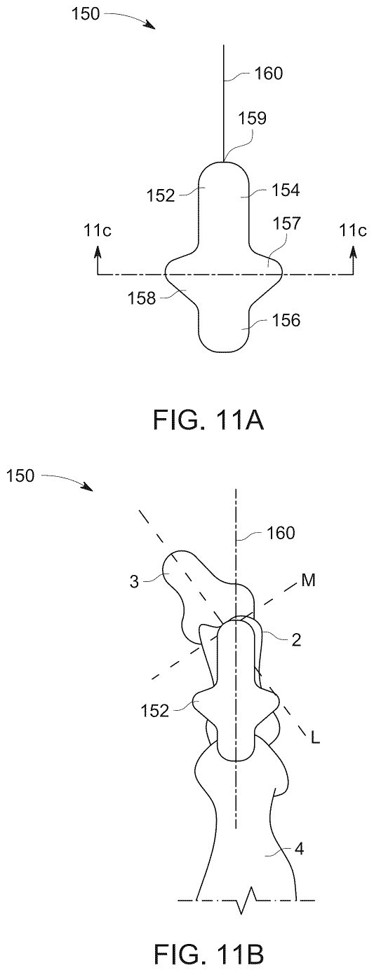

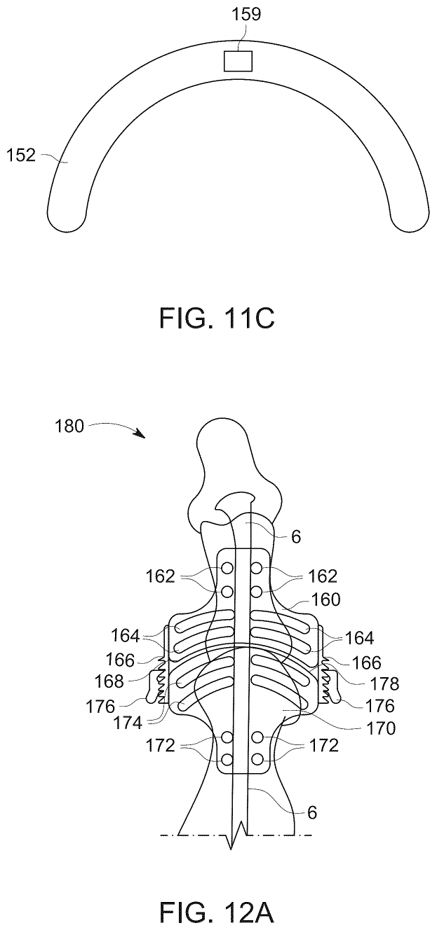

Turning now to FIGS. 10, 11A, 11B, and 11C, there is illustrated a targeting guide 150 configured to assist in treating hallux valgus interphalangeus (HVI) or high hallux interphalangeal angle (HIA). Hallux valgus interphalangeus is a deformity of the big toe characterized by an abnormal (valgus) angulation between the proximal and distal phalanx bones 2, 3 forming the end of the big toe. It is generally considered present if the two end bones (proximal and distal phalanx 2. 3) form an angle exceeding 10 degree. Both deformities are characterized by the hallux deviating laterally so that the joint lines of the interphalangeal joint and the MTP joint are misaligned and not in a substantially parallel relationship in the transverse plane.

In high HIA cases, joint resection is done in an uncoupled fashion, i.e., the metatarsal head and the proximal phalanx are resected and prepared independently of one another.

In HVI cases, the targeting chip 152 is placed such that the longitudinal axis of the chip is positioned perpendicular to the HVI deformity. The targeting guide chip 152 has an axial reference pin 160 extending from a central longitudinal axis of the targeting guide chip. Resection of the proximal phalanx 2 is then performed and the targeting guide chip 152 is aligned with the axial reference pin such that it is perpendicular to the hallux interphalangeal joint alignment and not perpendicular to the base of the proximal phalanx 2 in a neutral alignment.

In HVI and HIA cases, the targeting guide chip 152 also, optionally, has a generally cruciform shape with a longitudinal axis with two longitudinally extending arms 154, 156 along the longitudinal axis and two laterally and medially extending arms 157, 158 substantially perpendicular to the longitudinal axis of the targeting guide chip. A recess opening 159 is positioned at a distal end of the targeting guide chip configured to receive an alignment rod or pin 160 that is positioned mid-line to the targeting guide chip 152. In this manner, the proximal and distal phalanx bones 2, 3 may be drilled in an offset manner to correct the interphalangeal joint alignment characteristic of the HVI and HIA deformities.

The plantar surface of the targeting guide chip may, optionally, have a concave surface configuration to nest against the skin of the interphalangeal joint.

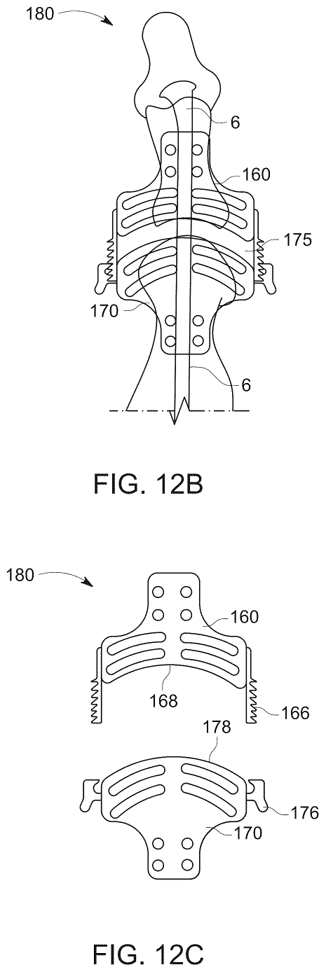

Another variant of a targeting guide 180 is illustrated in FIGS. 12A-12C, which depicts a multi-planar cut guide for MIS-MTP joint fusion. According to targeting guide variant, the targeting guide 180 is a two piece construct in which a first guide member 160 is configured to be placed on the dorsal skin surface of the 1^{st} metatarsal bone and the medial eminence of the 1^{st} metatarsal bone to guide cutting the articular surface of the 1^{st} metatarsal bone. A second guide member 170 is configured to be positioned proximal toe the first guide member 160. The first guide member 160 has a body (not numbered), a distal body portion at a distal end which includes bores 162, a main body portion including a plurality of opposing arched pin holes 164 and 166, and a curved surface 168 at the proximal end of the main body portion. The first guide member 160 has first engagement members 166 configured to engage second engagement members 176 of the second guide member 170, as shown in FIGS. 12A and 12B. The second guide member 160 includes a distal body portion with bores 172, a main body portion with arched pin holes 176, 178, and engagement members 176. The metatarsal chip is centered with a guidewire at the level of the MTP joint and has a cutting guide opening to guide burr cutting of the articular surface of the 1^{st} metatarsal.

A medial wing of the first guide member 160 is provided with at least one opening to guide placement of a proximal-medial to distal lateral fixation screw into the MTP joint without interfering with an adjacent interfragmentary screw. The distal dorsal aspect of the first guide member 160 has arched pin holes 164 for anatomic placement and pinning the MTP joint to ensure appropriate anatomic alignment of the joint.

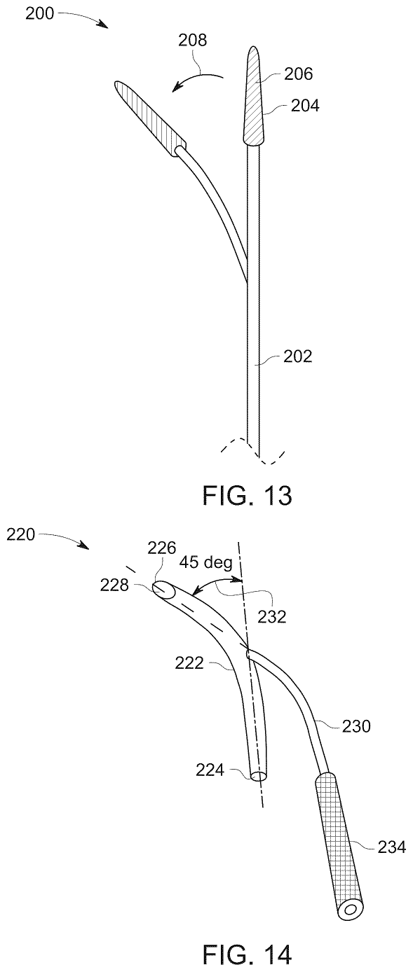

FIGS. 13 and 14 depict a MIS flexible bit 200 and a reamer passer 220, respectively. The MIS flexible reamer bit 200 consists of a flexible wire 202 having a diameter of between about 1.2 mm to about 3.0 mm and a tapered reamer 204 at a distal end of the wire 202. The tapered reamer 204 may be integral with or coupled to a distal end of the wire and may have a diameter larger than the wire diameter. The tapered reamer preferably has external threading 206 to facilitate drilling and removal of bone marrow back into the joint. The flexible wire 202 is preferably about 10 cm to about 16 cm in length and is removably attachable to a drill.

The reamer passer 220 has a curved wire sheath 230 that has a curvature of between about 30 degrees to about 45 degrees to concomitantly redirect a flexible reamer bit at a similar angle so that the reamer bit is generally perpendicular to the bone surfaces and aid in entering the bone without travel or skiving off the bone. The curved wire sheath 230 may have a longitudinal taper such that a distal end of the wire sheath 230 has a smaller diametric opening, which may, optionally, be tapered to a point to engage the bone surface to be drilled. A handle 234 is preferably attached to the wire sheath to allow the surgeon to control the positioning of the curved wire sheath.

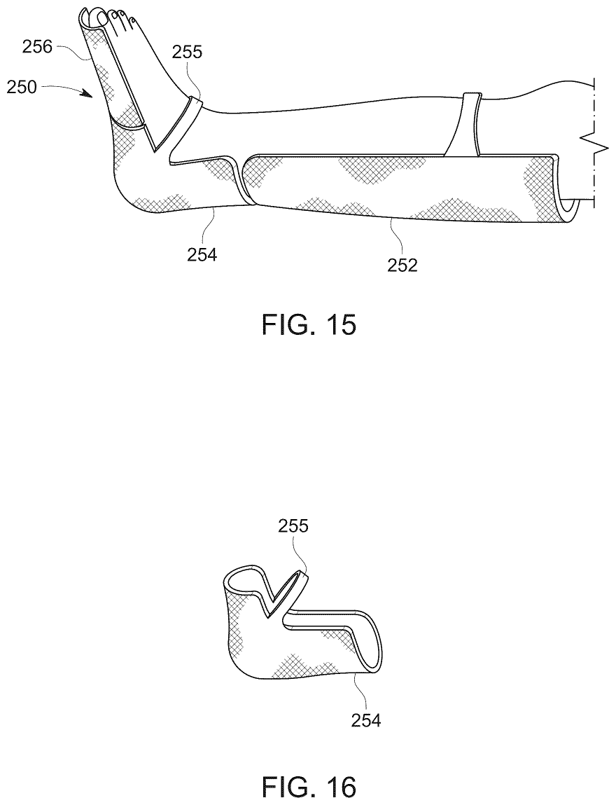

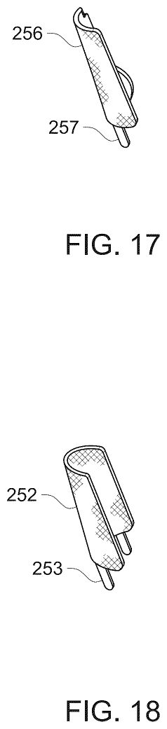

With reference to FIGS. 15 to 18, there is illustrated a modular leg, midfoot, and forefoot positioner device (hereinafter “modular positioner device”) 250. The modular positioner device 250 is configured to secure the leg, midfoot and forefoot of a patient during a surgical procedure. Owing to its modular design, the modular positioner device may be configured for just mid-foot positioning, just mid-foot and forefoot positioning, just mid-mid-foot, forefoot, and 1^{st} toe positioning, and/or leg, mid-foot and optionally forefoot and toe positioning. FIG. 15 illustrates the entire below need positioning with the mid-foot positioner 254, the forefoot positioner 252 and the 1^{st} toe positioner 256 assembled. FIG. 16 illustrate the mid-foot and heel cup as well as the open mesh construct of the mid-foot and heel cup section with a securing strap 255 that extends from the lateral to the medial surfaces of the mid-foot and heel cup section to secure the section to the patient. The forefoot component 252 illustrated in FIG. 18 has attachments 253 that attach to the mid-foot and heel cup section, such as hook-and-loop material or straps or the like, is positioned on the plantar surface of the foot and extends from the mid-foot to the forefoot and may extend to secure the toes. Illustrated in FIG. 17 is the 1^{st} toe positioner component 256, which is a modular component to assist in positioning the 1^{st} toe for a surgical procedure. The 1^{st} toe positioner has at least one attachment 257, such as hook-and-loop material or straps or the like, that is removably joinable with either the heel component, the mid-foot and heel component and/or the forefoot component and abuts the plantar surface of the 1^{st} toe.

All components of the modular positioner device 250 have an open mesh construct with mesh openings configured to permit the surgeon to pass fixation screws and/or wires through the mesh and into the foot anatomy. The open mesh construct may be made of plastic, fabric, composite or similar material having sufficient rigidity and pliability to be molded to the shape of the leg and foot, and stabilize the positioning of the foot and/or toes.

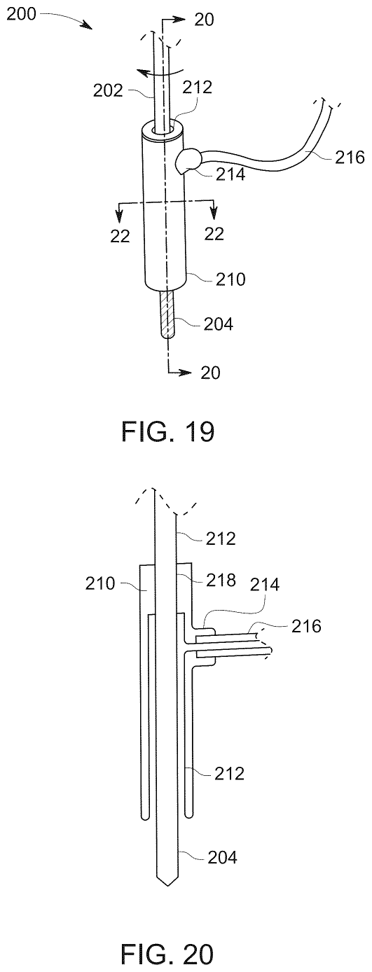

FIGS. 19 to 24 depict an alternative embodiment of a burr sleeve 200 that delimits the exposed depth of a cutting bur or a reamer bit 204 to limit the cut depth. The burr sleeve 200 is also configured to allow withdrawal of ground cartilage, bone, or bone marrow through a lumen of the burr sleeve under vacuum and/or injection of pharmacologically active agents, such as bone growth drugs, e.g., Teriparatide, Abaloparatide, Romosozumab, or bisphosphonate drugs, or fluids into the reamed opening through the lumen of the burr sleeve.

The burr sleeve consists generally of a hollow sleeve of wear resistant material 210 that fits over a burr 204 to limit depth penetration of the burr. The burr sleeve 200 is sized to stop against the targeting chip on a distal end of the burr sleeve and against the chuck of the burr driver on the proximal end of the burr sleeve. Different length burr sleeves allow for different burr lengths and different maximum depths of penetration; depth of penetration may be adjusted by adjusting the length of the burr relative to the burr driver chuck.

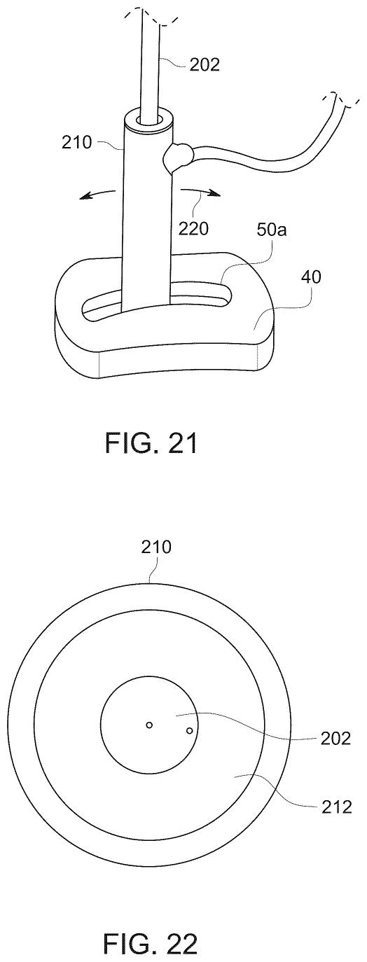

The burr sleeve has a narrow bore 202 at its proximal end to accommodate the reamer bit and burr 204 to pass through along a longitudinal axis of the burr sleeve 200 and project from a distal end of the burr sleeve 200. According to one variant of the burr sleeve, the narrow bore communicates with a larger diameter bore 212 toward the distal end of the burr sleeve. A port 214 passes through a side wall of the burr sleeve and is in fluid flow communication with the larger diameter bore 212 to facilitate application of a vacuum to withdraw ground cartilage, bone and/or bone marrow during burring and to facilitate injection of pharmacologically active agents or other fluids into the bone and/or joint. FIG. 21 illustrates use of the burr sleeve on a reamer bit in limiting the penetration depth of the burr (not shown) by abutting against the targeting guide as the reamer bit passes into a cutting guide in the targeting guide.

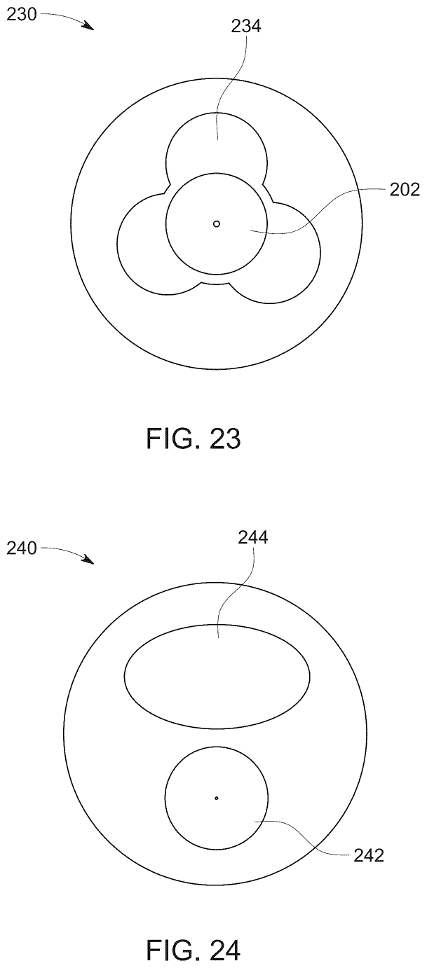

FIG. 22 is a transverse cross-sectional view of the above-described first variant illustrating the central co-axial positioning of the reamer bit in the large diameter bore 212 of the burr sleeve 210. FIG. 23 is illustrated a second variant of the burr sleeve 230 in which there is a multi-lobed central bore 234 within the burr sleeve with the reamer bit 202 passing co-axially within the central bore leaving the multi-lobed openings as tissue or fluid channels for withdrawal or injection of substances. FIG. 24 illustrates a multi-lumen variant of the burr sleeve 240 in which one lumen 242 is configured for the reamer bit and another lumen 244 is present for withdrawal or injection of substances.

The foregoing written specification is considered to be sufficient to enable one skilled in the art to practice the invention. The present disclosure is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize or be able to ascertain using no more than routine experimentation, many equivalents to the specific embodiments of the invention described herein. Such equivalents are intended to be encompassed by the following claims.

1-15. (canceled)

16. A targeting guide, comprising:
a chip having proximal and distal aspects thereof, the chip having a medial or lateral surfaces thereof configured to approximate a medial or lateral skin surface over a joint or bone; and
at least two openings passing through the medial or lateral surfaces and a thickness of the chip, each of the at least two openings having different angular opening axes and planes.

17. The targeting guide according to claim 16, wherein:
the joint or bone is a metatarsal bone of a first metatarsal-phalangeal joint; and
the chip further comprises an opening on the distal aspect thereof and a wire coupling the chip to a mid-line bore in one of the metatarsal bone or a phalanx bone.

18. The targeting guide of claim 17, further comprising a channel on a plantar surface of the chip configured to engage and isolate an extensor hallucis longus ligament therein.

19. The targeting guide of claim 17, wherein the angular opening axes and planes of the at least two openings are configured to create drilling and affixation screw bores that pass through the metatarsal bone and into a proximal phalanx bone without intersecting or interfering with each other.

20-27. (canceled)

28. The targeting guide according to claim 16, wherein:
the joint or bone is a proximal phalanx bone of a first metatarsal-phalangeal joint; and
the chip further comprises an opening on the distal aspect thereof and a wire coupling the chip to a mid-line bore in the proximal phalanx bone.

29. The targeting guide of claim 28, further comprising a channel on a plantar surface of the chip configured to engage and isolate an extensor hallucis longus ligament therein.

30. The targeting guide of claim 28, wherein the angular opening axes and planes of the at least two openings are configured to create drilling and affixation screw bores that pass through the proximal phalanx bone and into a metatarsal bone without intersecting or interfering with each other.

31-32. (canceled)

33. The targeting guide according to claim 16, wherein the chip further comprises an opening on the distal aspect thereof and a wire coupling the chip to a mid-line bore in a proximal.

34. The targeting guide according to claim 16, further comprising a channel in a surface of the chip configured to engage and isolate an at least one of a muscle, tendon, or ligament therein.

35. The targeting guide according to claim 16, wherein the angular opening axes and planes of the at least two openings are configured to create drilling and affixation screw bores that pass through a first bone and into a second bone without intersecting or interfering with each other.

36-43. (canceled)

44. An interphalangeal joint targeting guide comprising:
a main body member having a longitudinal axis and a targeting pin projecting distally from the main body member along a mid-line of the main body member and co-axial with the longitudinal axis of the main body member.

45. The interphalangeal joint targeting guide according to claim 44, wherein the main body member has a generally cruciform shape.

46. The interphalangeal joint targeting guide of claim 45, wherein the main body member has a concave plantar surface configured to nest against skin covering a proximal phalanx.

47. The interphalangeal joint targeting guide of claim 44, wherein the main body member has a concave plantar surface configured to nest against skin covering a proximal phalanx.

48. A targeting guide comprising:
a main body having a longitudinal axis, a first and second longitudinal extensions, and a first and second lateral extensions, and the main body further defines a plurality of elongate openings extending through the first and second longitudinal extensions from a dorsal surface to a plantar surface thereof.

49. The targeting guide of claim 48, wherein the main body has a generally cruciform shape.

50. The targeting guide of claim 48, wherein the plurality of elongate openings include a first set of elongate openings and a second set of elongate openings, the first set being parallel to the longitudinal axis, and the second set being perpendicular to the longitudinal axis.

51. The targeting guide of claim 50, wherein the first set of the plurality of elongate openings are configured to allow a fixation wire to be placed through an opening of the first set to secure the main body to both a distal aspect of a first metatarsal bone and a proximal aspect of a phalangeal bone while allowing longitudinal adjustment of the main body relative to the longitudinal axis.

52. The targeting guide of claim 51, wherein the second set of the plurality of elongate openings are configured to allow different size burrs to pass into and through an opening of the second set to allow surgical access to articular surfaces and ends of each of the first metatarsal bone and the phalangeal bone.

53. The targeting guide of claim 48, wherein a first set of the plurality of elongate openings have graduated opening widths or graduated angular positions relative to the longitudinal axis to permit cutting of different curvatures in each of a metatarsal bone and a phalangeal bone.
